(19) United States
(12) Patent Application Publication
(10) Pub. No.: US 2004/0211423 A1
(43) Pub. Date: Oct. 28, 2004

(54) Method for controlling the differential pressure in a CPAP device and CPAP device

(76) Inventor: Martin Baecke, Dessau (DE)

Correspondence Address: KING & SCHICKLI, PLLC, 247 NORTH BROADWAY, LEXINGTON, KY, 40507, US

(21) Appl. No.: 10/852,827

(22) Filed: May 25, 2004

Related U.S. Application Data
(63) Continuation of application No. PCT/DE02/04534, filed on Dec. 11, 2002.

(30) Foreign Application Priority Data
Dec. 11, 2002 (DE) 101 61 057.2-44

(51) Int. Cl. A61M 15/00; A61M 16/00

(52) U.S. Cl. 128/204.23; 128/204.18; 128/200.24

(57) ABSTRACT

The present invention relates to methods for controlling the differential pressure in a CPAP device, wherein the differential pressure is adjusted in response to the measured ambient air pressure and/or the measured ambient temperature so as to compensate said environmental influences during the therapy. Moreover, the present invention relates to CPAP device comprising an additional ambient air pressure sensor and/or an ambient temperature sensor.

### CROSS REFERENCE TO RELATED CO-PENDING APPLICATIONS

This application is a continuation of international PCT application number PCT/DE02/04534 (publication number: WO 03/049793 A2) filed on Dec. 11, 2002 and entitled METHOD FOR CONTROLLING THE DIFFERENTIAL PRESSURE IN A CPAP DEVICE AND CORRESPONDING CPAP DEVICE, which claims priority to German patent application number 101 61 057.2-44 filed on Dec. 11, 2002 and entitled VERFAHREN ZUR STEUERUNG DES DIFFERENZDRUCKS IN EINEM CPAP-GERÄT SOWIE CPAP-GERÄT the contents of which are expressly incorporated herein by reference.

### FIELD OF THE INVENTION

The present invention relates to a method for controlling the differential pressure in a CPAP device in response to an ambient air pressure and/or the ambient temperature. This invention further relates to a CPAP device which controls the speed of the turbine in response to an ambient air pressure and/or the ambient temperature.

### BACKGROUND OF THE INVENTION

The CPAP (continuous positive airway pressure) therapy was developed for the treatment of apneas during the sleep and is described in Chest. Volume No. 110, pages 1077-1088, October 1996 and in Sleep, Volume No. 19, pages 184-188. With apneas, the upper respiratory tract is contracted due to the relaxation of the muscles at the tongue root. Apneas result in respiratory complaints up to a respiratory standstill during the night.

A CPAP device is schematically illustrated in FIG. 1. It generates a positive overpressure up to approximately 30 mbar by means of a compressor or turbine 2 and administers the same, preferably via a humidifier, via a respiratory hose 4 and a nose and face mask 5 to the respiratory tract of the patient. This overpressure is to ensure that the upper respiratory tract remains fully opened during the whole night, so that no apneas will occur (DE 198 49 571 A1). The required overpressure depends, inter alia, on the sleeping stage and position of the body of the sleeping person.

The overpressure generated in the mask 5 will hereinafter be called therapeutic pressure ps. A typical CPAP device moreover comprises a pressure sensor 3 for measuring the overpressure generated by the turbine 2 in comparison to the ambient pressure, which is, in most cases, accommodated in the CPAP device. Moreover, a typical CPAP device comprises a control circuit, wherein the overpressure measured by the pressure sensor 3 is compared with a preadjusted target pressure and the speed of the turbine is controlled such that the measured pressure corresponds to the target pressure, if possible. One or more openings 7 cause air exhaled by the patient and enriched with CO₂ to be carried off into the environment, with the result that it is not enriched inside the respiratory hose 4.

It is desirable to provide a method and CPAP device which guarantee, on the one hand, a reliable therapy also under different environmental conditions and, on the other hand, keep the overpressure administered to the respiratory tract as low as possible.

### SUMMARY OF THE INVENTION

According to an embodiment of the invention a method for controlling the differential pressure in a CPAP device is provided. The differential pressure is the pressure difference between the pressure in the mask and the ambient air pressure. The ambient air pressure is measured. The differential pressure is adjusted in response to the measured ambient air pressure.

According to another embodiment of the invention another method for controlling the differential pressure in a CPAP device is provided. According to this method, the ambient temperature is measured. Finally, the differential pressure in response to the measured ambient temperature is adjusted.

According to a further embodiment of the invention a CPAP device is provided. The CPAP device comprises a turbine and a differential pressure sensor which measures the pressure difference between the overpressure generated by the turbine and the ambient air pressure.

The CPAP device further comprises a control device for controlling the speed of the turbine in response to the signal outputted by the differential pressure sensor. A second pressure sensor measures the ambient air pressure, wherein the signal delivered by the second pressure sensor is supplied to the control device and likewise influences the speed of the turbine.

According to yet a further embodiment of the invention a CPAP device is provided which comprises a turbine, a first pressure sensor and a control device which controls the speed of the turbine in response to the signal delivered by the pressure sensor. The CPAP device further comprises a second pressure sensor, wherein the first pressure sensor measures the absolute pressure generated by the turbine and the second pressure sensor measures the absolute ambient air pressure. The signal delivered by the second pressure sensor is supplied to the control device and likewise influences the control of the speed of the turbine.

According to another embodiment of the invention a CPAP device comprises a turbine, a pressure sensor and a control device for controlling the speed of the turbine in response to the signal delivered by the pressure sensor. The CPAP device further comprises a temperature sensor which measures the ambient temperature of the CPAP device, wherein the signal delivered by the temperature sensor is supplied to the control device and likewise influences the control of the speed of the turbine.

According to another embodiment of the invention a CPAP device comprises a turbine, a pressure sensor and a control device for controlling the speed of the turbine in response to the signal delivered by the pressure sensor. The CPAP device further comprises a temperature sensor which measures the temperature of the air transported by the turbine, wherein the signal delivered by the temperature sensor is supplied to the control device and likewise influences the control of the speed of the turbine.

An advantage in the adjustment of the differential pressure, i.e. also of the therapeutic pressure, in response to the measured ambient air pressure resides in that the therapeutic pressure is adapted to deviations in the ambient pressure. Deviations in the ambient air pressure may be caused by weather changes, i.e. from high pressure to low pressure areas and vice versa, or by trips to areas located at different levels in view of the sea level.

According to a simple method the differential pressure may linearly be dependent on the ambient pressure. However, also more complicated dependences may be chosen, e.g. according to equations (15) or (17) to (19).

An advantage in the adjustment of the differential pressure in response to the ambient temperature resides in that the influence of another ambient parameter is compensated. The differential pressure may, in a particularly advantageous manner, be adjusted in response to the measured ambient pressure as well as in response to the ambient temperature.

The common inventive concept between the adjustment of the differential pressure in response to the measured ambient air pressure and the adjustment of the same in response to the measured ambient temperature resides in the compensation of ambient influences.

### BRIEF DESCRIPTION OF THE DRAWINGS

Preferred embodiments of the invention will hereinafter be explained in more detail with reference to the attached drawings, wherein

FIG. 1 shows a CPAP device according to the invention, and

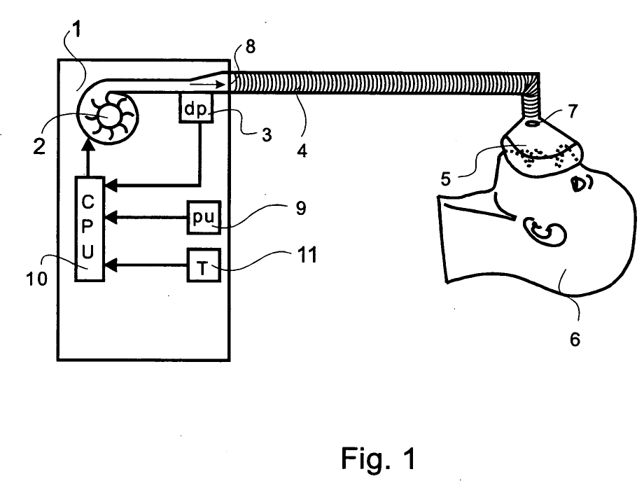

FIG. 2 shows a diagram, in which the conventional therapeutic pressure is compared with a therapeutic pressure according to a preferred embodiment of the present invention.

### DETAILED DESCRIPTION OF THE INVENTION

For gaining a model-like idea of the respiratory tract in the throat/larynx area the following model will be used: The first instable portion is located at the tongue root between the inherently rigid portions throat and larynx. For modelling the instable portion it is assumed that a negative pressure formed in the respiratory tract during inhalation acts, as compared with the ambient pressure, on the cross-section of the airway in a contracting manner. With a given muscle relaxation the effective cross-section is directly dependent on the pressure difference between the pressure in the respiratory tract and the ambient air pressure. Such a contraction of the cross-section of the airway can be counteracted by an overpressure in the respiratory tract as compared with the ambient air pressure, because the overpressure compensates the negative pressure formed in the respiratory tract during the inhalation at least partially. The dependence of the overpressure on the level of the ambient pressure will be discussed in the following.

Therefore, the pressure ps1 in the mask 5 is conventionally adjusted as a differential pressure dp as compared with the environment pu:

ps1=pu+dp (1)

Besides, the gravity acts onto the tissue, especially onto the tongue as part of the instable tissue. The gravitational influence is differently great in response to the sleeping position. If the patient sleeps on the back, the gravitational influence is greater as the gravity draws the tongue directly into the respiratory tract. If the patient lies on the side, the tongue falls primarily to the lower side of the mouth cavity. Only when the tongue deforms under the influence of the gravity and “flows” into the respiratory tract is the respiratory tract contracted and finally closed. In a side position the influence of the gravity is therefore smaller. For compensating the influence of the gravity an overpressure being substantially independent of the absolute ambient pressure is likewise necessary. The overpressure depends on the sleeping position, however.

Apart from the first instable portion there are additional instable portions located inside inherently rigid portions such as throat and larynx. By this, the form and position thereof is independent of the ambient pressure. Therefore, an absolute pressure pa, i.e. a pressure not related to the ambient pressure pu is required to press said additional instable portions out of the respiratory tract.

ps2=pa (2)

The gravity also acts on said additional instable portions. If necessary, the influence thereof, too, is treated by the absolute pressure pa.

Seen in the direction of the breath flow, the respiratory tract is formed of successively located sections with first instable portions and with additional instable portions, i.e. portions acted upon by the ambient pressure and those not acted upon by the ambient pressure. If ps1=pu+pd is high as against pa, it can be expected that ps1 must be adjusted as therapeutic pressure because this pressure also presses open said additional instable portions. If, vice versa, ps1 is lower than pa, pa has to be adjusted as therapeutic pressure.

Both influences can be taken into account by the empirically found calculation formula (3) for the determination of the necessary mask pressure ps:

ps=a*ps1+(1−a)*ps2 (3)

with 0<a<1, whereby the range 0.5<a<0.8 is particularly favourable. With a=1, the calculation is performed according to the conventional variant according to equation (1), with a=0 according to equation (2).

The breathing model may be further subtilized. To this end, it is assumed that the sleeping person has to inhale the same number of oxygen molecules per time unit. This corresponds to a constant mass flow \dot{m} of oxygen molecules (equation (4)). \dot{m} can be calculated as mean value of the breath volume by one or more breath cycles or as peak value of one breath cycle, or in any other way. This will not change anything in view of the qualitative results.

\dot{m}=const. (4)

On the other hand, the mass flow m can be calculated as integral over the surface A of the respiratory tract according to equation (5).

$$\dot{m} = {\int \int }_{A} ⅆ \dot{m} {\int \int }_{A} ρ v ( \vec{r} ) ⅆ A \qquad ( 5 )$$

d\dot{m} is a mass flow element per the element of surface dA. ρ is the density of the air, i.e. approximately 1.2 Kg/m³. v(\vec{r})is the vectorial speed component of the mass flow element d\dot{m} perpendicular to the element of surface dA.

For discussing the influence of different geometry parameters, different simplifications may be assumed for the speed distribution and the geometry of the respiratory tract. Under the assumption of a fully turbulent flow and a cylindrical geometry of the respiratory tract with a radius b/2, the following mass flow is obtained:

\dot{m}=ρ·v₀·(b/2)²·π (6)

v₀ thereby is the speed of the flowing medium and π is 3,14 . . . . Should the speed of the flowing medium not be constant with sufficient exactness, v₀ is the speed averaged over the surface A.

Under the assumption of a laminar flow the mass flow can be calculated with the aid of the Hagen-Poiseuille Law:

$$\dot{m} = \frac{π \cdot ρ \cdot Δ p \cdot {( b / 2 )}^{4}}{8 η \cdot l} \qquad ( 7 )$$

Δp is thereby the pressure drop at a portion of the respiratory tract assumed to be cylindrical with length l. η is the viscosity of the air.

Furthermore, the nearly closed respiratory tract can be assumed, shortly before an apnea, as a cuboid with length l, width b and height h. The air flows along length l and perpendicularly to width b and height h. The width b thereby be small as compared to the height h, so that the respiratory tract leaves open a slit-shaped opening. The speed distribution of the flowing air is parabolic over width b and—except for the marginal areas—constant over height h. The following formula refers to the mass flow:

$$\dot{m} = \frac{ρ \cdot Δ p \cdot h \cdot {b}^{3}}{4 η \cdot l} \qquad ( 8 )$$

According to equations (6), (7) and (8) the mass flow \dot{m} is always proportional to the density ρ of air. On the other hand, the mass flow depends on the characteristic opening of the respiratory tract b. For equations (6) and (7) the same is a radius, and in equation (8) the width of a slit. If other assumptions on the geometry of the respiratory tract are made, the characteristic expansion may be another dimension. If the smallest expansion of the respiratory tract is, for example, assumed to be an ellipse, the characteristic expansion is the smaller radius of the ellipse. The following empirical equation can be obtained from equations (6), (7) and (8):

$$\dot{m} = C \cdot \frac{ρ}{η} \cdot {b}^{e} \qquad ( 9 )$$

C is herein a constant comprising the dependences of v₀, h and/or l. Equation (9) shows that the mass flow \dot{m} is inversely proportional to the viscosity η. The characteristic expansion b appears with its e^{th} power. An expected range between 2 and 4 results for e from equations (6), (7) and (8).

The density ρ is proportional to the absolute air pressure p:

$$ρ = \frac{\overline{m} \cdot p}{k \cdot T} \qquad ( 10 )$$

Strictly speaking, p thereby is the air pressure at the narrowest point in the respiratory tract. p is approximately most likely equal to ps. \overline{m} is a suitably averaged mass between the weight of oxygen and nitrogen atoms and additional atoms and molecules existing in the air. k is the Boltzmann's constant and T the absolute temperature. Equation (10) results from the equation of state of ideal gases as can, for example, be found in F. Reif, Statistische Physik und Theorie der Wärme, de Gruyter, Berlin, 1987, 3^{rd} edition. It can moreover be inferred from this book that the viscosity η is independent of the density or the pressure of an ideal gas. This refers approximately also to air.

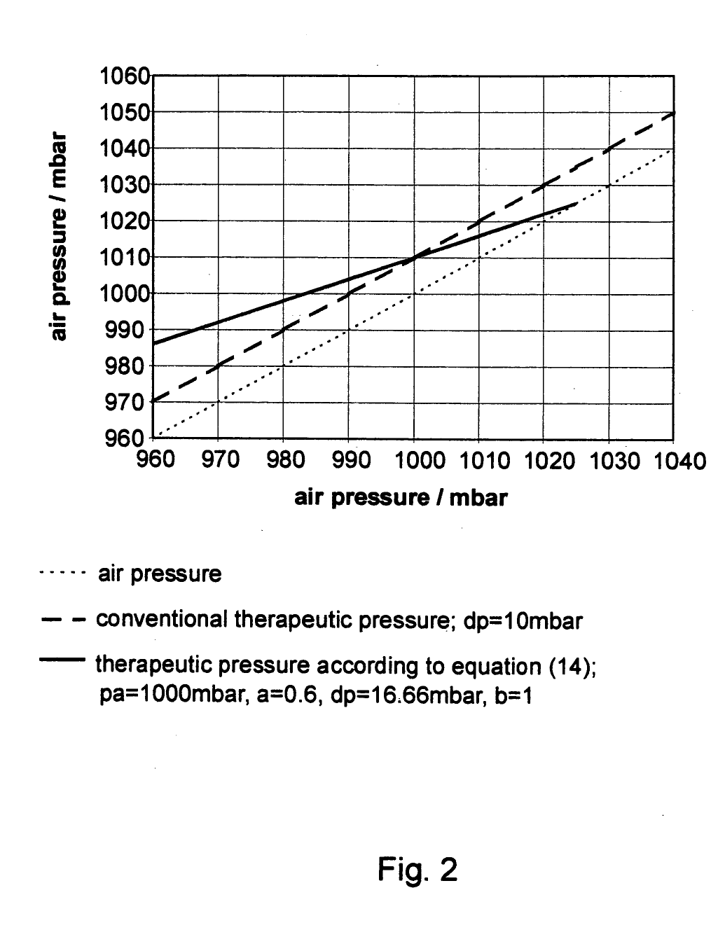

Consequently, the mass flow \dot{m} deviates proportionally to the absolute air pressure. Under the hypothesis proposed in equation (4), according to which the patient requires a constant mass flow \dot{m}, the characteristic opening of the respiratory tract b must be larger at a small air pressure p to allow the patient to breathe without problems. In view of quality the above-outlined model explains that, at a small air pressure p, the differential pressure dp, i.e. the overpressure in the respiratory tract, must be higher so as to widen the respiratory tract more strongly, as compared to a high air pressure. This relationship can also be inferred from FIG. 2.

In equation (9) it should be noted that not only the air pressure p, but also the viscosity η is dependent on the temperature. According to Reif (loc. cit.)

η=C_{η}\sqrt{T} (11)

C_{η} is thereby a proportional constant which is independent of the temperature.

If one puts equations (10) and (11) in (9) for b, one obtains:

$$b = {( \frac{\dot{m} \cdot k \cdot T \sqrt{T}}{C \cdot {C}_{η} \cdot \overline{m} \cdot p} )}^{1 / e} \qquad ( 12 )$$

b thereby has the meaning of a lower limit for the characteristic opening of the respiratory tract. Therefore, the equal sign of equation (12) may also be replaced by a ≧ sign. The required differential pressure d\check{p} can be expressed as function f of the characteristic opening b. The function f is developed into a power series and equation (11) is put in:

d\check{p}=f(b)≈a₀+a₁·b=a₀+\check{a}·p^{-1/e}·T^{3/2e} (13)

The constants a₀, \check{a} and e thereby constitute constants which are adapted to the therapeutic requirements in a suitable manner. Said constants may also be chosen in dependence on the sleeping state of the patient such that the differential pressure is adjusted as low as possible, but as high as necessary.

By the empirical adaptation of the constants a₀, \check{a} and e also the influence of muscles relaxing while the person is failing asleep, the supporting effect of surrounding cartilage and bones as well as—to a limited extent—the non-linear extensibility of the surrounding tissues is taken into account.

d\check{p} thereby is the differential pressure in the respiratory tract as compared to the ambient pressure. This is approximately the overpressure to be generated by the CPAP device as compared to the ambient pressure. By a small change of the constants a₀, \check{a} and e the pressure drop at the respiratory hose 4 can, for example, be taken into account in equation (13). Instead of the air pressure p in the respiratory tract either the pressure in the respiratory mask ps or even the ambient air pressure pu may be used, as said pressure values deviate from one another by approximately only 2%. This deviation can be compensated by a suited selection of the constants \check{a}. Eventually the exponents of pressure p and temperature T can also be adapted to the therapeutic requirements independently of each other, as is provided in equations (17) to (20). The air pressure in the mask is obtained from equation (13) by adding the ambient pressure to d\check{p}.

Due to the non-linear extensibility of the tissues of the upper respiratory tract and the square to cubical dependence of the pressure loss in the upper respiratory tract on the free cross-section thereof, and due to equation (13) an even better adaptation of the therapeutic pressure to the patient's needs can be achieved by introducing exponents c and i into equation (3):

ps=a·(pu+dp)^{C}+(1−a)·paⁱ (14)

with 0<c, i<2, preferably 0.8<c, i<1.2. The term (1−a)*paⁱ will thereby only be required for the adjustment of the pressure values in the sleeping laboratory. In the CPAP device itself the term can be stored as a constant value pa′, so that the pressure calculation is there accomplished according to a simplified formula:

ps=a·(pu+dp)^{C}+pa′ (15)

ps was the therapeutic pressure in the nose or face mask of the patient. A particular advantage of this method resides in that a reaction on changed environmental conditions of the patient can take place in an adequate manner. Essential are thereby deviations in the air pressure which depend on the weather and the altitude. The natural air pressure deviations move in a range of more than 60 mbar width by 1000 mbar. Under otherwise the same conditions this entails mass flow deviations of ±6%. In addition, there is a dependence on the height. In dependence on both the weather and the location, e.g. during a vacation or business trips, the air pressure changes far more than the differential pressure d\check{p}=ps−pu to be commonly adjusted by the CPAP device, which is smaller than 20 mbar in most cases.

It can be assumed that, in case of atmospheric high pressure conditions or stays on levels clearly lower than usual, the patient will sleep with a small preliminary pressure or even without a CPAP device quietly and without the appearance of any apnea. In case of an atmospheric low pressure condition or a stay in the mountains, however, there is the risk that an obstructive sleeping apnea occurs despite the use of a CPAP device, if the control is performed according to equation (1). These defects can effectively be counteracted by means of the method according to the invention.

The influence of the ambient temperature T still is to be discussed in the following. It can be inferred from equations (9) and (10) that the ambient temperature is put into the mass flow inversely proportionally. In addition, the viscosity η is proportional to the root from the temperature. On the whole, the following dependence results, with C₁ being a proportional constant:

$$\dot{m} = \frac{{C}_{1}}{T \sqrt{T}} \qquad ( 16 )$$

The typical ambient temperature during the sleep is 17° C., i.e. 290 K. In summer, in areas close to the equator, the ambient temperature may rise to 27° C. during the sleep, i.e. 300 K. Vice versa, the ambient temperature in winter, in areas close to the poles, may fall to below 7° C., i.e. 280 K. Thus, there are temperature deviations by ±3%, which again results in mass flow deviations of ∓4.5%. The same are smaller than the mass flow deviations caused by the pressure deviations, but reach the same order of magnitude. Also, the mass flow deviations caused by the pressure and temperature deviations will, most likely, frequently counteract each other, as both the temperature and the pressure decrease with an increasing height above sea level. This does not apply in general, however.

Therefore, an embodiment of a CPAP device according to the invention comprises a temperature sensor. The differential pressure is calculated in response to the ambient temperature.

In the following, some modifications of the equations for the calculation of the mask pressure ps will be described. Even though the equations are not equivalent from a mathematical point of view, a similar progression is achieved when the constants are chosen in a suited manner. All variables are thereby freely adaptable to the therapeutic requirements, except for the ambient pressure pu and the ambient temperature T. Equation (17) is similar to equation (15), but the ambient temperature T can be taken into account by the term g·(T−T₀)^{h} when the mask pressure ps is calculated.

ps=a·(pu+dp)^{C}+g·(T−T₀)^{h}+pa′ (17)

Equation (18) is similar to equation (17), however, by considering equation (13), the temperature dependence is taken into account by factor (T−T₀)^{h}.

ps=a·(pu+dp)^{C}·(T−T₀)^{h}+pa′ (18)

Equation (19) constitutes a simplified version of equation (17), whereby the constants g, h and T₀ flow into the constant pa′.

ps=a·pu^{C}+g·T^{h}+pa′ (19)

Equation (20) results from equation (13), whereby according to equation (20) the exponents of the ambient pressure pu and the ambient temperature T can be chosen independently of each other, and not the overpressure as compared to the ambient pressure, but the absolute pressure ps in the mask is calculated:

ps=a·pu^{C}·T^{h}+pa′ (20)

Conventional CPAP device comprise a microcontroller for controlling the speed of the turbine. The output signal of a differential pressure sensor is supplied to said microcontroller. The differential pressure sensor typically measures the differential pressure in the proximity of the respiratory hose connection 8 as compared to the ambient pressure and thus—by neglecting the pressure drop on the respiratory hose 4—also the overpressure in the mask 5. Embodiments of a CPAP device according to the invention moreover comprise an absolute pressure sensor 9 and/or an ambient temperature sensor 11. A sensor signal or both sensor signals are likewise digitalized and supplied to the microcontroller 10. Due to equation (13) the microcontroller can then calculate a desired differential pressure and control the speed of the turbine such that said desired differential pressure is also measured by the differential pressure sensor 3.

In other embodiments the mask pressure ps can at first be calculated on the basis of equations (17) to (19), from which results the desired differential pressure by deducting the ambient pressure pu.

In another embodiment of the CPAP device according to the invention the pressure sensors 3 and 9 constitute absolute pressure sensors. According to this embodiment the desired pressure ps is calculated from equations (17) to (20). In this embodiment, the central processing unit 10 controls the speed of the turbine such that the pressure measured by the pressure sensor 3 corresponds to the calculated desired pressure ps. By suitably choosing the constants in equations (17) to (20) the medium pressure drop on the respiratory hose can be compensated.

Equations (17) to (20) may be further simplified by suitably choosing the constants. If exponents c and h equal to 1 are chosen, the exponents need not be explicitly indicated in the equations and the exponentiation need not be performed. By choosing the exponents c or h equal to 0, the pressure or temperature dependence, respectively, disappears.

At present, it is expected from CPAP device that they allow the exact adjustment of the overpressure up to 0.1 mbar and that they maintain the overpressure just as exactly. This demand appears to be easier due to the use of one differential pressure sensor 3 and one absolute pressure sensor 9, i.e. it can be fulfilled at a lower price as compared to two absolute pressure sensors 3 and 9. In order to be able to exactly measure the overpressure at 0.1 mbar and to adjust the same, two absolute pressure sensors likewise both have to have an exactness of about 0.1 mbar in a measuring range of 1100 mbar, i.e. a relative error of 0.01%. If one absolute pressure sensor and one differential pressure sensor are used, the differential pressure gauge must have, in a measuring range of ±30, an exactness of below 0.1 mbar, i.e. a relative error of 0.3%. The absolute pressure sensor has, as compared to the differential pressure gauge, a smaller influence on the overpressure. In the example explained on the basis of FIG. 2, an absolute pressure change of 25 mbar results in an overpressure change of about 10 mbar, so that the absolute pressure sensor should have a relative exactness of 0.025%. Thus, if an absolute pressure sensor is used with a differential pressure sensor, higher sensor tolerances may be accepted, with the consequence that more inexpensive sensors can be employed.

In the foregoing, the invention was explained in more detail by means of preferred embodiments. For a person skilled in the art it is obvious, however, that different modifications may be made, without deviating from the spirit of the invention. Therefore, the scope of protection is defined by the following claims and the equivalents thereof.

List of Reference Numbers

1 CPAP device

2 turbine

3 differential pressure sensor

4 respiratory hose

5 face or nose mask

6 patient

7 throttle valve

8 respiratory hose connection

9 pressure sensor

10 central processing unit

11 temperature sensor

What is claimed is:

1. A method for controlling the differential pressure in a CPAP device, wherein the differential pressure is the pressure difference between the pressure in the mask and the ambient air pressure, comprising:
measuring the ambient air pressure; and
adjusting the differential pressure in response to the measured ambient air pressure.

2. The method according to claim 1, wherein the differential pressure depends on the sum of a constant pressure plus ambient pressure.

3. The method according to claim 2, wherein the differential pressure depends on the sum multiplied by a factor unequal to 1.

4. The method according to claim 3, wherein a constant pressure is added to the sum multiplied by a factor.

5. The method according to claim 2, wherein the sum is exponentiated with an exponent unequal to 1.

6. The method for controlling the differential pressure in a CPAP device, wherein the differential pressure is the pressure difference between the pressure in the mask and the ambient air pressure, comprising:
measuring the ambient temperature; and
adjusting the differential pressure in response to the measured ambient temperature.

7. The method according to claim 6, wherein the differential pressure is calculated from the difference of the measured temperature minus a constant temperature, wherein the difference is exponentiated with an exponent and this result is multiplied by a factor and added to a constant pressure.

8. A CPAP device, comprising:
a turbine,
a differential pressure sensor measuring the differential pressure between the overpressure generated by the turbine and the ambient air pressure;
a control device for controlling the speed of the turbine in response to the signal outputted by the differential pressure gauge,
a pressure sensor measuring the ambient air pressure, wherein the signal delivered by the pressure sensor is supplied to the control device and likewise influences the speed of the turbine.

9. The CPAP device according to claim 8, wherein the control device controls the speed of the turbine in response to the sum of a constant pressure plus ambient pressure.

10. The CPAP device according to claim 9, wherein the control device controls the speed of the turbine in response to the sum multiplied by a factor unequal to 1.

11. The CPAP device according to claim 9, wherein the sum is exponentiated with an exponent unequal to 1.

12. The CPAP device according to claim 8, wherein a temperature sensor for measuring the ambient temperature of the CPAP device, wherein the signal delivered by the temperature sensor is supplied to the control device and likewise influences the control of the speed of the turbine.

13. The CPAP device according to claim 8, wherein a temperature sensor for measuring the temperature of the air supplied by the turbine, wherein the signal delivered by the temperature sensor is supplied to the control device and likewise influences the control of the speed of the turbine.

14. A CPAP device, comprising:
a turbine,
a first pressure sensor, and
a control device for controlling the speed of the turbine in response to the signal delivered by the pressure sensor,
a second pressure sensor, wherein the first pressure sensor measures the absolute pressure generated by the turbine and the second pressure sensor measures the absolute ambient air pressure, the signal delivered by the second pressure sensor is supplied to the control device and likewise influences the control of the speed of the turbine.

15. The CPAP device according to claim 14, wherein the control device controls the speed of the turbine in response to the sum of a constant pressure plus ambient pressure.

16. The CPAP device according to claim 15, wherein the control device controls the speed of the turbine in response to the sum multiplied by a factor unequal to 1.

17. The CPAP device according to claim 15, wherein the sum is exponentiated with an exponent unequal to 1.

18. The CPAP device according to claim 14, wherein a temperature sensor for measuring the ambient temperature of the CPAP device, wherein the signal delivered by the temperature sensor is supplied to the control device and likewise influences the control of the speed of the turbine.

19. The CPAP device according to claim 14, wherein a temperature sensor for measuring the temperature of the air supplied by the turbine, wherein the signal delivered by the temperature sensor is supplied to the control device and likewise influences the control of the speed of the turbine.

20. The CPAP device, comprising:
a turbine,
a pressure sensor, and
a control device for controlling the speed of the turbine in response to the signal delivered by the pressure sensor,
a temperature sensor for measuring the ambient temperature of the CPAP device, wherein the signal delivered by the temperature sensor is supplied to the control device and likewise influences the control of the speed of the turbine.

21. The CPAP device according to claim 20, wherein the control device controls the speed of the turbine based on the difference of the measured temperature minus a constant temperature, wherein the difference is exponentiated with an exponent and this result is multiplied by a factor and added to a constant pressure.

22. The CPAP device, comprising:
a turbine,
a pressure sensor, and
a control device for controlling the speed of the turbine in response to the signal delivered by the pressure sensor,
a temperature sensor for measuring the temperature of the air transported by the turbine, wherein the signal delivered by the temperature sensor is supplied to the control device and likewise influences the control of the speed of the turbine.

23. The CPAP device according to claim 22, wherein the control device controls the speed of the turbine based on the difference of the measured temperature minus a constant temperature, wherein the difference is exponentiated with an exponent and this result is multiplied by a factor and added to a constant pressure.
